(19) United States
(12) Patent Application Publication
(10) Pub. No.: US 2019/0357768 A1
(43) Pub. Date: Nov. 28, 2019

(54) Glaucoma Surgery Visualization Apparatus

(71) Applicant: University of Rochester, Rochester, NY (US)

(72) Inventor: Shakeel Shareef, Rochester, NY (US)

(73) Assignee: University of Rochester, Rochester, NY (US)

(21) Appl. No.: 16/332,785

(22) PCT Filed: Sep. 11, 2017
(86) PCT No.: PCT/US17/51029

§ 371 Date: Mar. 12, 2019

Related U.S. Application Data
(60) Provisional application No. 62/393,627, filed on Sep. 12, 2016.

(51) Int. Cl. A61B 3/117; A61B 3/125

(52) U.S. Cl. CPC A61B 3/117; A61F 9/00781; A61B 3/125

(57) ABSTRACT

There is provided a glaucoma surgery visualization apparatus. The apparatus has a goniolens which is rotationally coupled to a novel scleral retention device. The apparatus allows for viewing of the irido-corneal angle structures of the eye and related surgical procedures. The scleral retention device uses vacuum to temporarily adhere the apparatus to the sclera. Surgical apertures allow for surgical access, while the goniolens can rotate within the scleral retention device to facilitate proper viewing of the irido-corneal angle structures before, during and after surgery. An optional light source serves to illuminate the field of viewing to further assist the surgeon during various procedures that involve the glaucoma surgery visualization apparatus.

### CROSS REFERENCE TO RELATED PATENT APPLICATIONS

This application claims priority to U.S. Patent Application Ser. No. 62/393,627 filed Sep. 12, 2016 entitled “Glaucoma Surgery Visualization Apparatus” by Dr. Shakeel Shareef. M.D., and to International Application Number PCT/US17/51029 filed Sep. 11, 2017 entitled “Glaucoma Surgery Visualization Apparatus”, the entire disclosures of which are incorporated herein by reference in their entirety.

### BACKGROUND OF THE INVENTION

### 1. Field of the Invention

The present invention relates generally to examination and surgery of the eye, and more particularly to a visualization apparatus for examination and surgery of the irido-corneal angle structures of the eye.

### 2. Description of Related Art

The visualization of the irido-corneal angle structures of the eye is important to various diagnostic and surgical procedures such as laser trabeculoplasty, canaloplasty, drainage implants, and the like. In recent years, there have been tremendous advances in micro-invasive glaucoma surgery such as trabecular micro-bypass stents. While such surgical procedures and related devices have advanced the field of glaucoma treatment, the optical and surgical instruments used for such specialized surgeries has not kept pace with these advances in surgical treatment and related devices.

Gonioscopy is a technique widely used to visualize the irido-corneal angle structures of the eye. A goniolens is used in conjunction with a microscope during surgery in order to visualize the angle structures before and during surgery. Without such a device, these angle structures are not visible. The goniolens is an optical lens that contains a prism and associated optics to allow for viewing of the angle structures of the eye. Koeppe and Goldmann pioneered goniolenses in 1919 and 1938, with many advances taking place since their inception.

While the goniolens is an incredibly useful optical instrument, it places a burden on the surgeon as it requires retention and stabilization by the surgeon using the non-surgical hand while performing surgical procedures with the dominant surgical hand. This two hand approach is cumbersome and requires a level of manual dexterity and skill that is beyond that of many otherwise experienced surgeons. In addition, the use of a gonioscope with one hand and surgical instruments in the other can create a potential for unwanted movements as well as hand fatigue, all of which are detrimental to efficient and safe surgical procedures.

What is needed is an apparatus to allow visualization of the irido-corneal angle structures of the eye without the need for placement and retention by the non-surgical hand, thus freeing up both hands for procedural use. What is also needed is an apparatus that not only provides such visualization, but also allows for stabilization and surgeon controlled movement of the eye during surgical procedures.

It is thus an object of the present invention to provide a visualization apparatus for examination and surgery of the irido-corneal angle structure of the eye while stabilizing the eye in a surgically optimal position and freeing up the non-dominant hand for other surgical uses. These and other objects of the present invention are not to be considered comprehensive or exhaustive, but rather, exemplary of objects that may be ascertained after reading this specification with the accompanying drawings.

### BRIEF SUMMARY OF THE INVENTION

In accordance with the present invention, there is provided a glaucoma surgery visualization apparatus comprising a goniolens rotationally coupled to a scleral retention device. Surgical apertures are provided to allow for surgical access. The goniolens is capable of rotating within the scleral retention device. The scleral retention device uses vacuum to temporarily adhere the apparatus to the sclera, thus also stabilizing the eye in a surgically optimal position and freeing up the non-dominant hand for other surgical uses. An optional light source serves to illuminate the field of viewing to further assist the surgeon and clinician during various procedures that benefit from the visualization apparatus.

The foregoing paragraph has been provided by way of introduction, and is not intended to limit the scope of the invention as described by this specification, claims and the attached drawings.

### BRIEF DESCRIPTION OF THE DRAWINGS

The invention will be described by reference to the following drawings, in which like numerals refer to like elements, and in which:

FIG. 1 is a perspective view of the glaucoma surgery visualization apparatus in use;

FIG. 2 is an exploded view of the glaucoma surgery visualization apparatus;

FIG. 3 is a top plan view of the glaucoma surgery visualization apparatus;

FIG. 4 is a side plan view of the glaucoma surgery visualization apparatus;

FIG. 5 is a bottom plan view of the glaucoma surgery visualization apparatus;

FIG. 6 is a bottom perspective view of the glaucoma surgery visualization apparatus;

FIG. 7 is a perspective view of a goniolens of the present invention;

FIG. 8 is a top plan view of the goniolens housing of the present invention;

FIG. 9 is a perspective view of the goniolens housing of the present invention;

FIG. 10 is a plan view of the rotational bezel of the present invention;

FIG. 11 is a perspective view of the rotational bezel of the present invention;

FIG. 12 is a top plan view of the glaucoma surgery visualization apparatus without the goniolens and goniolens housing;

FIG. 13 is a side plan view of the glaucoma surgery visualization apparatus without the goniolens and goniolens housing;

FIG. 14 is a perspective view of the glaucoma surgery visualization apparatus without the goniolens and goniolens housing;

FIG. 15 is a plan view of a rotational ring of the present invention; and

FIG. 16 is a rotational tool of the present invention.

The present invention will be described in connection with a preferred embodiment, however, it will be understood that there is no intent to limit the invention to the embodiment described. On the contrary, the intent is to cover all alternatives, modifications, and equivalents as may be included within the spirit and scope of the invention as defined by this specification, claims and drawings attached hereto.

### DESCRIPTION OF THE PREFERRED EMBODIMENTS

A glaucoma surgery visualization apparatus is described. The apparatus comprises a goniolens rotationally coupled to a novel scleral retention device. While the apparatus has tremendous utility in the field of glaucoma related surgeries, other surgeries and visualization procedures that require viewing of the irido-corneal angle structures of the eye along with retention and control of the eye will also benefit from the apparatus of the present invention.

All components of the present invention are made from surgical grade materials that may include, for example, stainless steel, plastics, and glass. Components may be fabricated by way of casting, machining, molding, grinding, polishing, and the like. While the apparatus described herein and depicted in the figures suggests a preferred embodiment, many variations and embodiments thereof may be envisioned after reviewing this specification and the accompanying drawings, such variations and embodiments being considered included in the present invention for which this disclosure pertains.

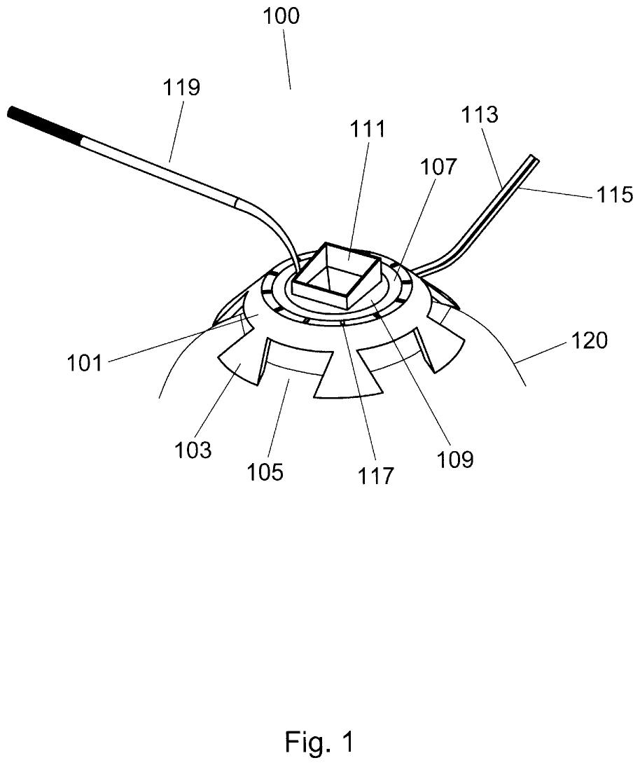

Turning now to the drawings, FIG. 1 is a perspective view of the glaucoma surgery visualization apparatus 100 in use and in place on an eye 120 of a patient. A scleral retention device 101 can be seen adhered to or otherwise placed on the sclera of the eye 120. As depicted, the eye 120 is not considered to be part of the present invention, but is shown as an example of the glaucoma surgery visualization apparatus 100 in use. The scleral retention device 101 has internal passageways that are connected to a vacuum line 113 to deliver negative pressure through a plurality of suction legs 103. The scleral retention device 101 may be made from a surgically acceptable material such as stainless steel, various plastics, and the like. The scleral retention device 101 may be molded, formed, pressed, machined, or otherwise fabricated. In some embodiments of the present invention, suction is delivered through a ring that is continuous or partially continuous, while other embodiments deliver suction through a plurality of suction legs 103 or other similar fixtures that allow the surgeon to selectively adhere and remove the scleral retention device 101 from the eye 120 during various surgical, examination, and related procedures. Each suction leg 103 has an angled surface that conforms to the sclera of the eye 120 just outside of the cornea in the perilimbal/scleral area, and may also be padded or otherwise lined with a conformal or soft durometer material to minimize any possible damage to the eye and also to improve suction if so required. The angled surface may be angled outward to conform to the eye and also may be curved to allow further conformity and vacuum adherence. The suction legs 103 may be square, rectangular, cylindrical, or the like and are hollow to provide for suction which is necessary to adhere the apparatus to the eye. The amount of suction provided through the vacuum line 113 can be varied by way of a valve or similar pressure regulator between the scleral retention device 101 and a source of vacuum such as a vacuum pump or the like. Between each suction leg 103 is a surgical aperture 105 that allows for placement and movement of surgical instruments such as probes, needles, inserters (such as inserters for trabecular micro-bypass stents), scalpels, and the like. In some embodiments of the present invention, the surgical aperture 105 is angled such that the opening becomes larger as one moves farther away from each suction surface of the suction leg 103. The scleral retention device 101 thus serves to retain a rotational goniolens or gonioprism to a patient's eye without the need for a surgeon to do so manually through the coupling or joining of the scleral retention device 101 to a goniolens by way of a housing or the like. A rotational bezel 107 can be seen between the scleral retention device 101 and the goniolens housing 109 to allow for rotational movement of the goniolens 111. The rotational bezel 107 may be made from a surgical grade metal, plastic, or the like, and may, in some embodiments of the present invention, contain a low coefficient of friction surface or component. The goniolens 111 is an optical device that is secured in a goniolens housing 109 which is then retained in a circular opening in the scleral retention device 101. A goniolens (or gonio lens or gonioscope) uses a lens and mirror or prism arrangement to gain a view of the iridocorneal angle (the angle formed between the eye's cornea and iris). Various goniolenses may be used to with the present invention, in fact in some embodiments of the present invention the goniolens may be interchangeable to suit the procedure. A series of markers 117 may, in some embodiments of the present invention, be placed around the scleral retention device 101 such that a surgeon can determine and mark the angular rotation of the goniolens 111 during various procedures. The markers 117 may, in some embodiments of the present invention, correspond to hour or minute placement on a clock, and may correspond to angular positions. To move the goniolens 111 with respect to the scleral retention device 101, a rotational tool 119 may be placed in a socket or opening in the goniolens housing 109 and moved in the desired direction. Also seen in FIG. 1, adjacent to the vacuum line 113 is an optional fiber optic line 115 that delivers light to the visualization apparatus at an appropriate location near the goniolens 111.

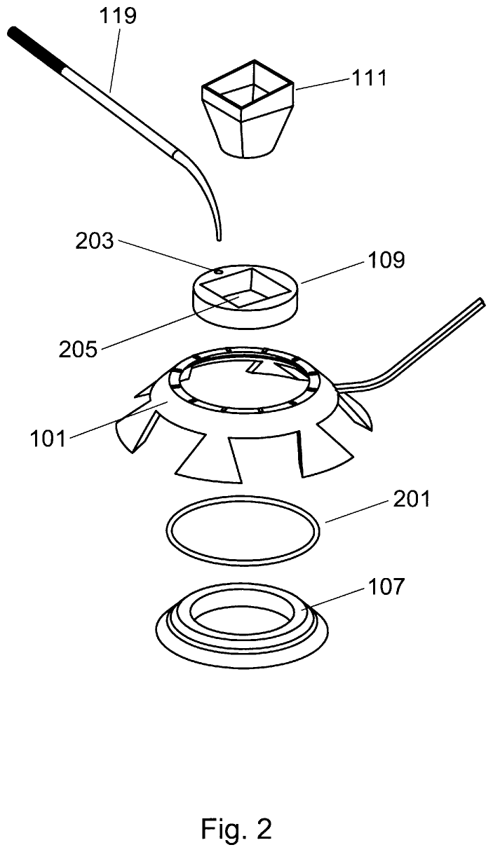

For a better understanding of the various constituent components of the glaucoma surgery visualization apparatus. FIG. 2 depicts an exploded view thereof. To provide retention and free movement of the goniolens 111 and associated goniolens housing 109 within the scleral retention device 101, the rotational bezel 107 can be seen with a rotational ring 201. The rotational ring 201 sits in a groove or a channel within the scleral retention device 101 and the rotational bezel 107, and provides retention and movement thereof. The rotational ring 201 may be made from a low friction plastic such as a polyethylene, a metal such as a stainless steel, or the like. The interworking of the rotational ring 201 and the rotational bezel 107 with respect to the scleral retention device 101 prevents the goniolens Ill and goniolens housing 109 from moving in a vertical direction orthogonal to the surface of the patient's eye, thus maintaining a fixed focal length for proper operation of the attached to goniolens 111. In some embodiments of the present invention, the rotational bezel 107 and the rotational ring 201 are one assembly or unit, or may be omitted entirely with the proper surface definition between the goniolens housing 109 and the scleral retention device 101.

It should be noted that a variety of goniolens or gonioprisms 111 may be used with the present invention with an appropriately sized goniolens housing 109. This provides for versatility in the selection of suitable goniolenses, gonioprisms, or other selected optics. The goniolens housing 109 retains a common exterior geometry and has a goniolens space 205 that is defined by the goniolens selected. This allows for most optics to be used with the present invention by making a housing 109 that is standard with respect to the scleral retention device 101 and related components. In some embodiments of the present invention, adapters, spacers or similar devices may be employed to provide further interchangeability between the goniolens 111 and the goniolens housing 109. In further embodiments, the goniolens housing 109 and the goniolens 111 may be one assembly or unit, or the goniolens housing 109 may be omitted entirely or in part given sufficient structural definition of the goniolens 111 and related goniolens packaging or components. A rotational tool socket 203 can also be seen along with the associated rotational tool 119. To rotate the goniolens 111 while the scleral retention device 101 remains affixed to the patient's eye, the rotational tool 119 is inserted in the socket 203 and the goniolens housing 109 and the attached goniolens 111 is moved in the required direction. Markers 117, as seen in FIG. 1, aid in the selection and recording of the selected viewing angle. The rotational tool 119, as will be further described herein, is removable such that it does not interfere with surgical procedures.

It should be noted that while the examples provided herein describe a rotational tool and socket with associated manual rotation, in some embodiments of the present invention the rotation may be provided by way of a small motor, such as a small servo or stepper motor, piezoelectric motor, or the like. Such an electric motor can then be controlled by way of a remote interface such as a surgical mouse or joystick arrangement, glove based sensors, eyewear with imaging controls, or the like. The control interface may also be an interface or control to robotically assisted surgery methods and systems.

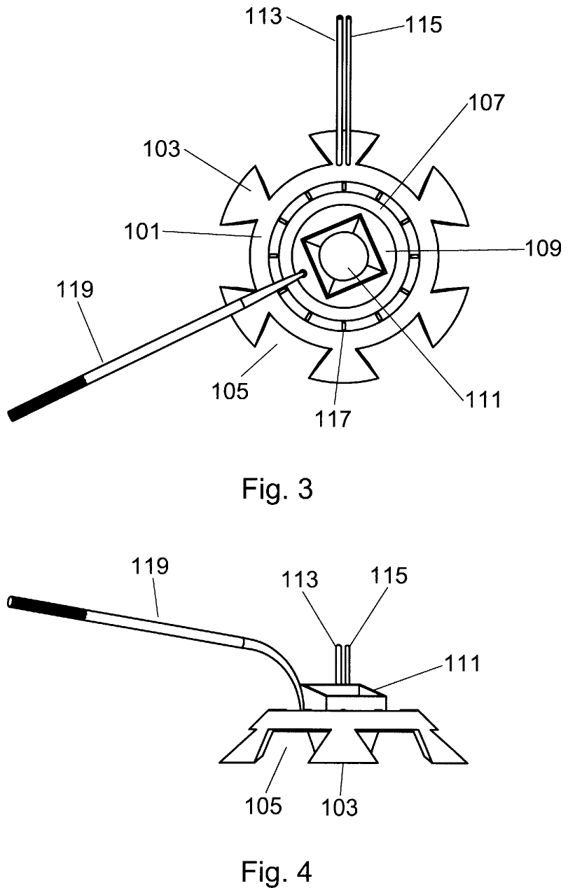

FIG. 3 is a top plan view of the glaucoma surgery visualization apparatus with the rotational tool 119 in place and in use or ready for use. In some embodiments of the present invention, a stop or frictional brake prevents unwanted rotation of the goniolens housing 109 and attached goniolens 111.

Both the vacuum line 113 and the fiber optic line 115 are depicted in the drawings without a connection. The vacuum line 113 in use would be connected to a source of vacuum or suction, and the fiber optic line 115 in use would be connected to a light source. Also, other components and instruments that are used in surgery and visualization procedures with traditional goniolenses would also be employed; these components and instruments being known to medical practitioners who specialize in such eye related procedures, and as such, the use of the novel glaucoma surgery visualization apparatus would become evident to such a practitioner after reading this specification in view of the attached drawings.

FIG. 4 is a side plan view of the glaucoma surgery visualization apparatus showing the goniolens 111 protruding downward so that it may contact a patient's eye. Various lubricants and optical coupling compounds may be used to ensure good optical clarity between the goniolens 111 and the eye.

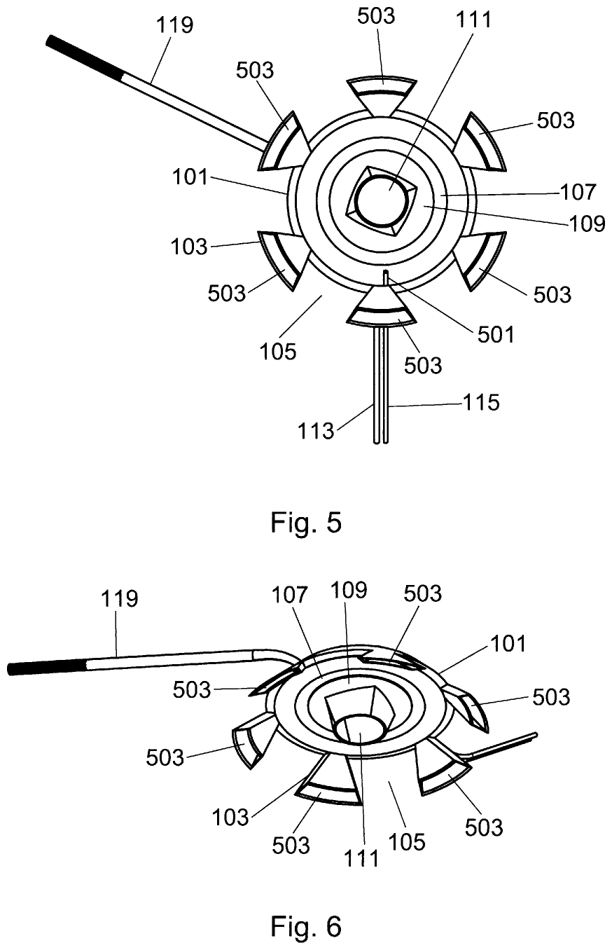

FIG. 5 is a bottom plan view of the glaucoma surgery visualization apparatus. The goniolens 111 can be seen in an arbitrary position that may be changed by the relevant movement of the rotational tool 119 when connected to the rotational tool socket 203 (as seen in FIG. 2). To illuminate the viewing angle achieved by the goniolens, a fiber optic lens 501 can be seen protruding from the underside of the glaucoma surgery visualization apparatus, and may be employed in some embodiments of the present invention. In a similar manner, a laser source may also be employed in addition to, or instead of, a white light source and related optics. The fiber optic lens 501 is optically coupled to the fiber optic line 115 that is in turn connected to a light source. The fiber optic lens 501 may be directional or omni-directional. In some embodiments of the present invention, the fiber optic lens 501 is capable of circumferential movement or angular movement to better direct the light from the fiber optic lens 501 onto the viewing subject. The fiber optic lens 501 may deliver full spectrum or select spectrum light for illumination purposes, and may also, in some embodiments of the present invention, deliver collimated (laser) light for some surgical procedures.

FIG. 6 is a bottom perspective view of the glaucoma surgery visualization apparatus. The suction ports 503 can be clearly seen at the eye contacting end of each suction leg 103. The suction ports 503, as depicted by the example in FIG. 6, may be curvilinear to better conform to a patient's eye, and may also be lined along the eye contacting perimeter with a soft durometer material to ensure that the sclera is not damaged while using the apparatus.

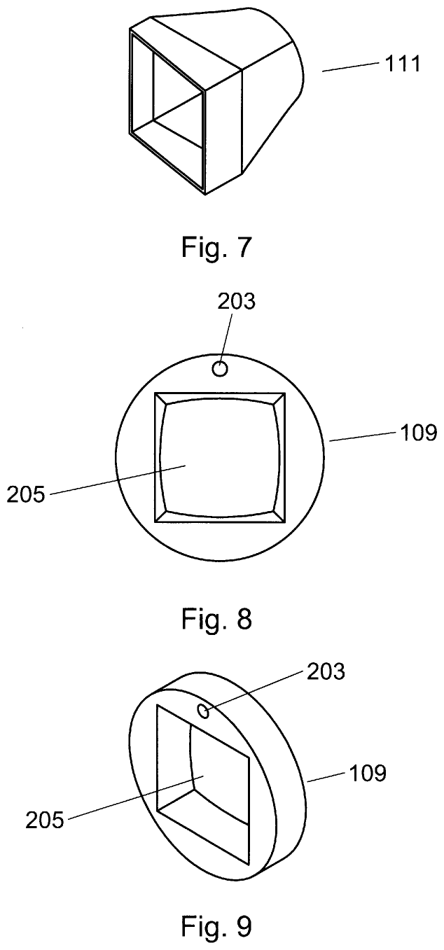

FIG. 7 is a perspective view of a goniolens of the present invention. The goniolens 111 depicted is merely exemplary, and not limiting in any way. Other goniolenses, gonioprisms, or a variety of optical elements may be employed with the present invention. Examples of surgical goniolenses include: Hoskins-Barkan, Ahmed, Mori, Double Mirror, Ritch, Hill (Right and Left), Swan Jacob, Osher Gonio and Post Pole, Khaw, Transcend Void Gonio Lens, as well as others. Manufacturers of goniolenses include, but are not limited to, Ocular Instruments, Inc. and Transcend Medical. Inc. The goniolens 111 is affixed to or otherwise placed in the goniolens housing 109 as seen in the top plan view of the goniolens housing depicted in FIG. 8. The goniolens 111 being placed in the space 205 and affixed either with pressure, adhesive, mechanical fasteners or attachments, and the like. The goniolens may also be moved within the space 205 to change the way in which the surface of the goniolens contacts the patient's eye.

FIG. 9 is a perspective view of the goniolens housing 109 of the present invention, which may be made from a surgical grade plastic, metal, or the like. The overall shape of the goniolens housing may vary based on the goniolens used for a particular application and any use of adapters, shims, or the like.

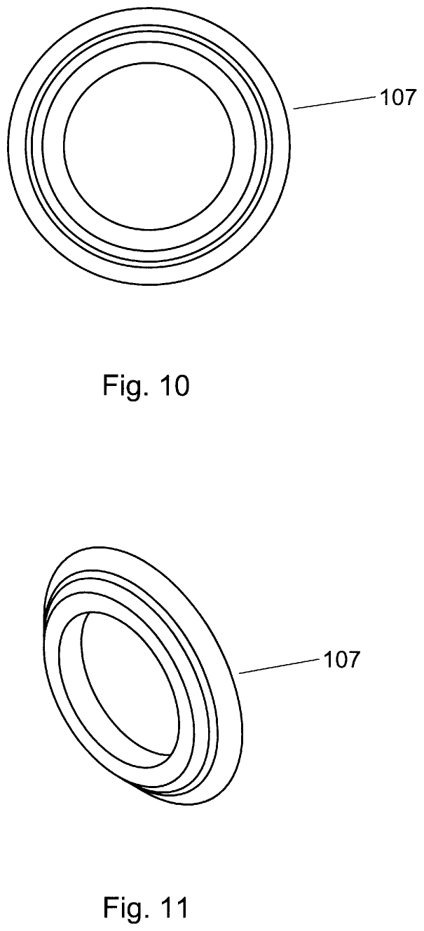

FIG. 10 is a plan view of the rotational bezel 107 of the present invention and FIG. 11 is a perspective view of the rotational bezel 107 of the present invention. The rotational bezel 107, as previously described, contains the goniolens housing 109 and the associated goniolens 111. The goniolens housing 109 is rotationally coupled to the rotational bezel 107, and may contain further fixtures such as the rotational ring 201, bearings, sleeves, guides, or the like.

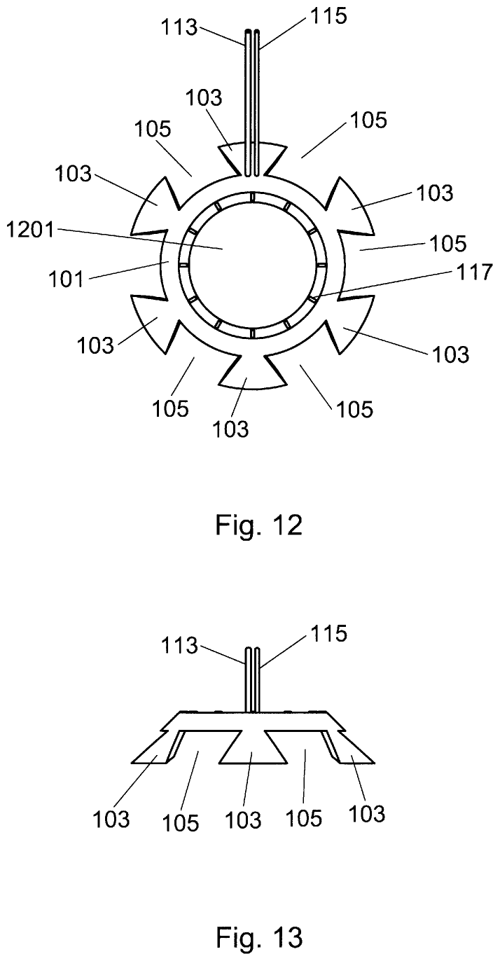

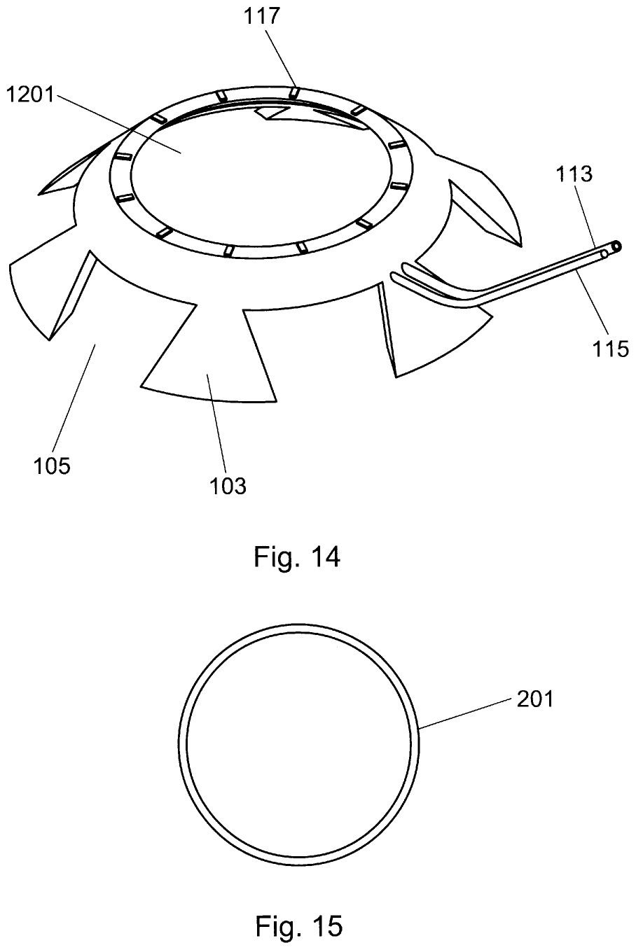

FIG. 12 is a top plan view of the glaucoma surgery visualization apparatus without the goniolens, goniolens housing, rotational bezel, or rotational ring. The rotational bezel 107, rotational ring 201, goniolens Ill and goniolens housing 109, each of which can be seen in FIG. 2, make up what is known as the rotational assembly. This rotational assembly is contained in the space 1201 as seen in FIG. 12. FIG. 13 is a side plan view of the glaucoma surgery visualization apparatus without the rotational assembly as previously described. FIG. 14 is a perspective view of the glaucoma surgery visualization apparatus without the rotational assembly.

FIG. 15 is a plan view of the rotational ring 201 of the present invention. The rotational ring 201 provides both retention of the rotational assembly as well as a sliding rotational surface to facilitate rotation of the goniolens 111 and associated goniolens housing 109. The interoperability of the rotational ring 201 with the other components of the glaucoma surgery visualization apparatus can be seen in FIG. 2.

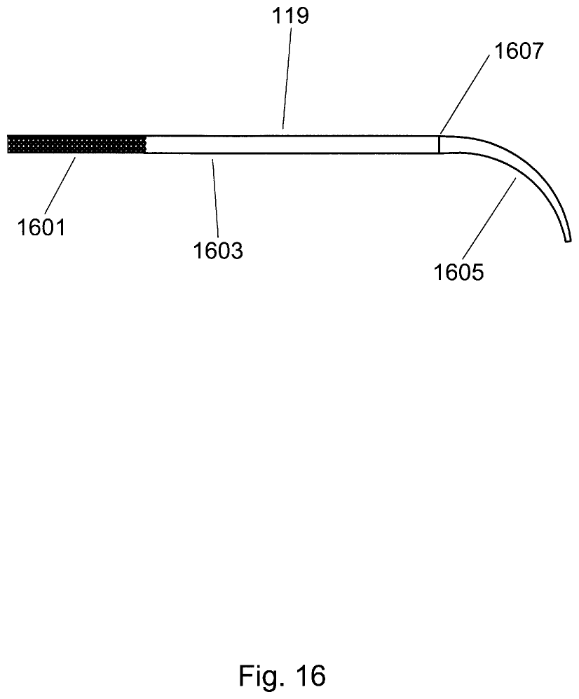

Lastly, FIG. 16 is a rotational tool 119 of the present invention. While the rotational tool 119 is depicted with a curved shaft, in some embodiments of the present invention, the shaft may be straight, angled, or of other geometries. A handle 1601 can be seen to facilitate retention by the surgeon. A shaft 1603 can also be seen that terminates in an engagement tip 1605. The engagement tip 1605 is placed in or otherwise coupled to the rotational tool socket 203 to facilitate rotation of the goniolens 111 within the scleral retention device 101. Optionally, a joint 1607 may be incorporated with the shaft 1603 to allow for a change of curvature, angle, or the like. The joint 1607 may be a universal joint, a hinged joint, or the like. The rotational tool 119 may be made from a surgically acceptable metal, plastic, or the like, and may be molded, cast, machined, or otherwise formed.

To use the glaucoma surgery visualization apparatus, the apparatus is placed on a patient's eye after a lubricant or optical coupling medium is placed between the interface of inferior aspect of the goniolens and where it contacts the eye. Vacuum is then activated to adhere the apparatus to the sclera, with positioning of the apparatus being optimized by the attending surgeon. Once stable, the goniolens may be used and rotated as necessary using the rotational tool 119 and associated rotational assembly. The irido-corneal angle may now be viewed by the surgeon without the use of the surgeon's non-dominant hand. The patient's eye is now stable, and can be moved to a surgically optimal position as necessary. Surgical procedures may be performed before, during, or after placement of the glaucoma surgery visualization apparatus. Surgical apertures 105 may be used to access the eye for some procedures. Vacuum may be changed to facilitate proper adhesion of the apparatus to the eye, and once angle viewing and related surgical procedures have been completed, the apparatus is removed from the eye after the vacuum has been turned off.

It is, therefore, apparent that there has been provided, in accordance with the various objects of the present invention, a glaucoma surgery visualization apparatus.

While the various objects of this invention have been described in conjunction with preferred embodiments thereof, it is evident that many alternatives, modifications, and variations will be apparent to those skilled in the art. Accordingly, it is intended to embrace all such alternatives, modifications and variations that fall within the spirit and broad scope of this specification, claims and drawings appended herein.

What is claimed is:

1. A glaucoma surgery visualization apparatus comprising:
a goniolens rotationally coupled to a scleral retention device for retention of a goniolens to the eye of a patient without the need for a surgeon to do so manually.

2. The glaucoma surgery visualization apparatus of claim 1, wherein the scleral retention device comprises vacuum passageways.

3. The glaucoma surgery visualization apparatus of claim 1, wherein the scleral retention device comprises a plurality of suction legs where each suction leg has a vacuum passageway making up a suction port and a surface that conforms to the sclera of the eye of a patient.

4. The glaucoma surgery visualization apparatus of claim 3, wherein space between adjacent suction legs forms a surgical aperture to allow for placement and movement of surgical tools.

5. The glaucoma surgery visualization apparatus of claim 1, further comprising a goniolens housing for securing the goniolens and a rotational bezel for providing rotational coupling of the goniolens housing to the scleral retention device.

6. The glaucoma surgery visualization apparatus of claim 1, further comprising a series of markers placed around the scleral retention device to allow a surgeon to determine and mark angular rotation of the goniolens with respect to the scleral retention device during a surgical procedure.

7. The glaucoma surgery visualization apparatus of claim 1, further comprising a rotational tool socket in the goniolens housing for placement of a rotational tool.

8. The glaucoma surgery visualization apparatus of claim 7, wherein the rotational tool comprises a rotational tool shaft with a rotational tool engagement tip configured to provide insertion of the rotational tool engagement tip into the rotational tool socket of the goniolens housing.

9. The glaucoma surgery visualization apparatus of claim 1, further comprising a fiber optic line for delivery of light and subsequent illumination of a surgical region.

10. The glaucoma surgery visualization apparatus of claim 9, further comprising a light source connected to the fiber optic line.

11. The glaucoma surgery visualization apparatus of claim 9, further comprising a fiber optic lens optically coupled to the fiber optic line.

12. The glaucoma surgery visualization apparatus of claim 1, wherein the goniolens housing comprises a goniolens space for receiving and retaining a variety of goniolenses.

13. The glaucoma surgery visualization apparatus of claim 1, further comprising a rotational ring to prevent the goniolens and the goniolens housing from moving in a vertical direction orthogonal to the surface of a patient's eye.

14. A glaucoma surgery visualization apparatus comprising:
a scleral retention device comprising a plurality of suction ports and a plurality of surgical apertures;
a goniolens retained by a goniolens housing and rotationally coupled to the scleral retention device.

15. The glaucoma surgery visualization apparatus of claim 14, wherein the scleral retention device conforms to the sclera of a patient's eye.

16. The glaucoma surgery visualization apparatus of claim 14, further comprising a rotational bezel for providing angular movement of the goniolens with respect to the scleral retention device.

17. The glaucoma surgery visualization apparatus of claim 14, further comprising a series of markers placed around the scleral retention device to allow a surgeon to determine and mark angular rotation of the goniolens with respect to the scleral retention device during a surgical procedure.

18. The glaucoma surgery visualization apparatus of claim 14, further comprising a fiber optic line for delivery of light and subsequent illumination of a surgical region.

19. The glaucoma surgery visualization apparatus of claim 14, further comprising a rotational coupling of the goniolens housing to the scleral retention device.

20. A glaucoma surgery visualization apparatus comprising:
a scleral retention device comprising a plurality of suction ports, a plurality of surgical apertures and a rotational tool socket;
a goniolens retained by a goniolens housing and rotationally coupled to the scleral retention device; and
a rotational tool comprising a handle, a shaft, and an engagement tip.
